(19) United States
(12) Patent Application Publication
(10) Pub. No.: US 2021/0406009 A1
(43) Pub. Date: Dec. 30, 2021

(54) APPARATUS FOR OPTIMIZED MICROCODE INSTRUCTIONS FOR DYNAMIC PROGRAMMING BASED ON IDEMPOTENT SEMIRING OPERATIONS

(71) Applicant: Western Digital Technologies, Inc., San Jose, CA (US)

(72) Inventor: Daniel BEDAU, San Jose, CA (US)

(21) Appl. No.: 16/917,634

(22) Filed: Jun. 30, 2020

(51) Int. Cl. G06F 9/30; G06F 9/38; G06F 9/48; G06F 17/10; G16B 30/10

(52) U.S. Cl. CPC G06F 9/3001; G06F 9/3836; G06F 9/48; G16B 30/10; G06F 9/3889; G06F 17/10; G06F 9/3887

(57) ABSTRACT

In one embodiments, a method is provided. The method includes determining whether a set of algorithmic operations can be represented using an algebraic formulation. The method also includes generating a sequence of idempotent semiring operations based on the set of algorithmic operations in response to determining that the set of algorithmic operations can be represented using the algebraic formulation. The sequence of idempotent semiring operations are part of an algebraic idempotent semiring, represent the algebraic formulation, and comprise one or more of an associative, commutative pick operation that forms an abelian monoid and an associative tally operation that forms a monoid and distributes over the pick operation. The method also includes generating a sequence of microcode instructions based on the sequence of idempotent semiring operations, wherein the sequence of microcode instructions carries out the sequence of idempotent semiring operations.

### BACKGROUND

### Field of the Disclosure

This disclosure relates to generating microcode operations for a processing device. More particularly, the disclosure relates to a generating microcode instructions for a processing device based on idempotent semiring operations.

### Description of the Related Art

There are various techniques/methods for solving different computational problems, such as finding the shortest or least expensive path in a graph of connected nodes. One such technique/method for solving a computational problem may be dynamic programming. Dynamic programming is a method/technique where a more complicated problem is broken down into simpler sub-problems in a recursive manner. The complicated problem may be solved by combining solutions to the simpler, overlapping, sub-problems.

### SUMMARY

In some embodiments, a method is provided. The method includes determining whether a set of algorithmic operations can be represented using an algebraic formulation. The method also includes generating a sequence of idempotent semiring operations based on the set of algorithmic operations and a set of idempotent semiring operations, in response to determining that the set of algorithmic operations can be represented using the algebraic formulation. The set of idempotent semiring operations are part of an algebraic idempotent semiring, represent the algebraic formulation, and comprise one or more of an associative, commutative pick operation that forms an abelian monoid and an associative tally operation that forms a monoid and distributes over the pick operation. The method also includes generating a sequence of microcode instructions based on the sequence of idempotent semiring operations, wherein the sequence of microcode instructions carries out the sequence of idempotent semiring operations.

In some embodiments, an apparatus is provided. The apparatus includes a memory and a processing device operatively coupled to the memory. The processing device is configured to determine whether a set of algorithmic operations of a dynamic programming algorithm can be represented using an algebraic formulation. In response to determining that the set of algorithmic operations can be represented using the algebraic formulation, the processing device is also configured to generate a sequence of idempotent semiring operations based on the set of algorithmic operations and a set of idempotent semiring operations. The set of idempotent semiring operations are part of an algebraic idempotent semiring. The set of idempotent semiring operations represent the algebraic formulation. The processing device is further configured to generate a sequence of microcode instructions based on the set of idempotent semiring operations. The sequence of microcode instructions carry out the set of idempotent semiring operations.

In some embodiments, a non-transitory machine-readable medium having executable instructions is provided. The executable instructions cause one or more processing devices to perform operations. The operations include determining whether a set of algorithmic operations can be represented using an algebraic formulation. The operations also include in response to determining that the set of algorithmic operations can be represented using the algebraic formulation, generating a sequence of idempotent semiring operations based on the set of algorithmic operations and a set of idempotent semiring operations. The set of idempotent semiring operations are part of an algebraic idempotent semiring. The set of idempotent semiring operations represent the algebraic formulation. The operations further include generating a sequence of microcode instructions based on the sequence of idempotent semiring operations. The sequence of microcode instructions carry out the sequence of idempotent semiring operations.

In some embodiments, an apparatus is provided. The apparatus includes a memory configured to store a sequence of microcode instructions. A subset of the sequence of microcode instructions are based on a set of idempotent semiring operations. The set of idempotent semiring operations are part of an algebraic idempotent semiring. The set of idempotent semiring operations represent an algebraic formulation representing a set of algorithmic operations. The apparatus also includes a hardware processing device operatively coupled to the memory and comprising a set of processing units. The processing device and/or set of processing units are configured to receive the sequence of microcode instructions. The sequence of microcode instructions carries out the set of idempotent semiring operations. The set of processing units are configured for parallelized operations based on one or more of the algebraic formulation and the set of idempotent semiring operations. The processing device and/or set of processing units are also configured to execute the sequence of microcode instructions in the set of processing units.

In some embodiments, a method is provided. The method includes obtaining a sequence of microcode instructions. A subset of the sequence of microcode instructions are based on a set of idempotent semiring operations. The set of idempotent semiring operations are part of an algebraic idempotent semiring. The set of idempotent semiring operations comprise one or more of an associative, commutative pick operation that forms an abelian monoid and an associative tally operation that forms a monoid and distributes over the pick operation. The set of idempotent semiring operations represent an algebraic formulation representing a set of algorithmic operations. The sequence of microcode instructions carries out the set of idempotent semiring operations. The method also includes executing the sequence of microcode instructions in a set of processing units of a hardware processing device. The set of processing units are configured for parallelized operations based on one or more of the algebraic formulation and the set of idempotent semiring operations.

### BRIEF DESCRIPTION OF THE DRAWINGS

FIG. 1A is a diagram illustrating example computing devices, in accordance with some embodiments of the present disclosure.

FIG. 1B is a diagram illustrating an example computing device, in accordance with some embodiments of the present disclosure.

FIG. 2 is a diagram illustrating an example systolic array, in accordance with some embodiments of the present disclosure.

FIG. 3 is a diagram illustrating an example instruction module, in accordance with some embodiments of the present disclosure.

FIG. 4 is a diagram illustrating an example graph, in accordance with some embodiments of the present disclosure.

FIG. 5 is a diagram illustrating an example decoder for decoding a bit stream, in accordance with some embodiments of the present disclosure.

FIG. 6 is a diagram illustration example DNA sequences, in accordance with some embodiments of the present disclosure.

FIG. 7 is a diagram illustrating example matrices, in accordance with some embodiments of the present disclosure.

FIG. 8 is a flowchart illustrating an example a process for generating microcode instructions, in accordance with one or more embodiments of the present disclosure.

FIG. 9 is a flowchart illustrating an example a process for executing microcode instructions, in accordance with one or more embodiments of the present disclosure.

FIG. 10 is a block diagram of an example computing device that may perform one or more of the operations described herein, in accordance with some embodiments of the present disclosure.

To facilitate understanding, identical reference numerals have been used, where possible, to designate identical elements that are common to the figures. It is contemplated that elements disclosed in one embodiment may be beneficially utilized on other embodiments without specific recitation.

### DETAILED DESCRIPTION

In the following disclosure, reference is made to examples, implementations, and/or embodiments of the disclosure. However, it should be understood that the disclosure is not limited to specific described examples, implementations, and/or embodiments. Any combination of the features, functions, operations, components, modules, etc., disclosed herein, whether related to different embodiments or not, may be used to implement and practice the disclosure. Furthermore, although embodiments of the disclosure may provide advantages and/or benefits over other possible solutions, whether or not a particular advantage and/or benefit is achieved by a given embodiment is not limiting of the disclosure. Thus, the following aspects, features, embodiments and advantages are merely illustrative and are not considered elements or limitations of the appended claims except where explicitly recited in a claim(s). Likewise, reference to “the disclosure” shall not be construed as a generalization of any inventive subject matter disclosed herein and shall not be considered to be an element or limitation of the appended claims except where explicitly recited in the claim(s).

The headings provided herein are for convenience only and do not necessarily affect the scope or meaning of the claimed invention. Disclosed herein are example implementations, configurations, and/or embodiments relating generating microcode instructions based on idempotent semiring operations.

As discussed above, there are various techniques/methods for solving different computational problems. One such technique/method may be dynamic programming, where a more complicated problem is broken down into simpler sub-problems in a recursive manner. The complicated problem may be solved by combining solutions to the simpler, overlapping, sub-problems. Writing programs to solve dynamic programming problems and executing these programs on general computing devices (e.g., general purpose processors) may be difficult for users (e.g., programmers). In order to write programs, applications, apps, etc., to solve dynamic programming problems, a user may factor in the type of hardware that is used and the user may have to parallelize the code manually to allow the program to execute faster.

In various embodiments, examples, and/or implementations disclosed herein, a set of algorithmic operations may represent a solution for a computational problem, such as a dynamic programming problem. A set and/or a sequence of idempotent semiring operations may be generated based on the set of algorithm operations. The use of idempotent semiring operations allows the dynamic programming problem to be represented using an algebraic formulation which may be bounded by a limited set of operations under a sequence of operations (e.g., bounded with operators that have pre-defined properties). The set and/or sequence of idempotent semiring operations may be converted into microcode instructions. The microcode instructions are generated such that they are easy to execute in parallel, since the order or sequence of operations in the formulation (along with specific properties (e.g., communicative) related to the operators) define what operations can be done in parallel and what operations need to follow an order or sequence. This decomposition into a formalistic expression enables ease of hardware efficiency tuning and parallelized execution. Efficiency can be gained also due to the limited number of idempotent semiring operations involved and hardware can be discretized or otherwise optimized for those operations. A hardware processing device with multiple processing units may be configured to execute the microcode instructions in parallel. The hardware processing device may be able to change modes/configurations to execute microcode instructions generated from different idempotent semiring operations that are part of different algebraic semirings. This allows the solution to a computational program to be defined using an algebraic representation. Prior knowledge of how the underlying hardware will execute instructions and can simply focus on formulating the problem using an algebraic representation. This allows separation of the execution and optimization of a program from the formulation of the computational program, which may allow optimized programs, applications, etc., more easily. This also allows the operation/execution of a program, application, etc., to be parallelized more easily.

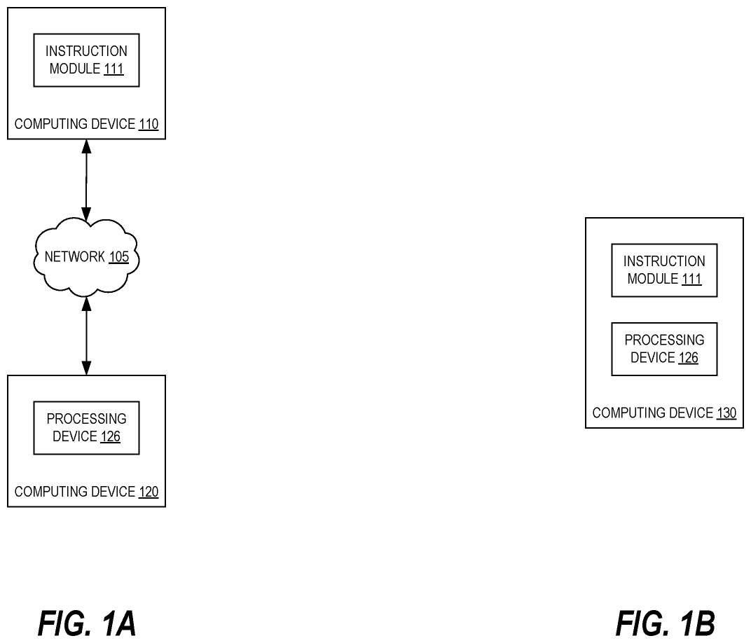

FIG. 1A is a diagram illustrating example computing devices 110 and 120, in accordance with some embodiments of the present disclosure. The computing device 110 and computing device 120 may be coupled to each other (e.g., may be operatively coupled, communicatively coupled, may communicate data/messages with each other) via network 105. Network 105 may be a public network (e.g., the internet), a private network (e.g., a local area network (LAN) or wide area network (WAN)), a data bus, or a combination thereof. In one embodiment, network 105 may include a wired or a wireless infrastructure, which may be provided by one or more wireless communications systems, such as a Wi-Fi hotspot connected with the network 105 and/or a wireless carrier system that can be implemented using various data processing equipment, communication towers (e.g. cell towers), etc. In some embodiments, the network 105 may be an L3 network. The network 105 may carry communications (e.g., data, message, packets, frames, etc.) between computing devices 110 and computing device 120.

Each of computing device 110 and computing device 120 may include hardware such as processing devices (e.g., processors, central processing units (CPUs), graphical processing units (GPUs), programmable logic devices (PLDs), processing units, data processing units (DPUs), a systolic array, processing units that broadcast/transmit data between each other, etc.), memory (e.g., random access memory (e.g., RAM), storage devices (e.g., hard-disk drive (HDD), solid-state drive (SSD), etc.), and other hardware devices (e.g., sound card, video card, etc.). Each computing device 110 and 120 may comprise any suitable type of computing device or machine that has a programmable processor including, for example, server computers, desktop computers, laptop computers, tablet computers, smartphones, set-top boxes, etc. In some examples, each of the computing devices 110 and 120 may comprise a single machine or may include multiple interconnected machines (e.g., multiple servers configured in a cluster). In the case of multiple interconnected machines, the tasks and functions described in the various examples below could be distributed and executed in those multiple machines in a coordinated manner. For simplicity of description, those tasks and functions will be generally described with respect to a single module.

Computing device 110 includes an instruction module 111. As discussed above, a solution to computational problem (e.g., an algorithm, a set of algorithmic operations) may be represented using an algebraic formulation. Instruction module 111 may determine a set and/or a sequence of idempotent semiring operations based on the set of algorithmic operations. The instruction module 111 may also generate microcode instructions that may perform the set and/or sequence of idempotent semiring operations when a processing device (e.g., processing device 126) executes the microcode instructions. The idempotent semiring operations may be part of an algebraic semiring (e.g., an algebraic idempotent semiring, as discussed in more detail below).

In one embodiment, the instruction module 111 may determine whether a set of algorithmic operations can be represented using an algebraic formulation. The algorithmic operations may include a set of operations, actions, that form a solution for a computational problem. As discussed above, one type of computational problem may be a dynamic programming problem. For example, dynamic programming problems include, but are not limited to, a maximum likelihood decoder (e.g., a Viterbi decoder), a maximum a posteriori decoder (e.g. the BCJR algorithm), aligning two sequences/strings (e.g., aligning two deoxyribonucleic acid (DNA) sequences/strings), finding the shortest or least expensive path in a graph of connected nodes, etc. The set of algorithmic operations may be an algorithm (e.g., a set of operations/actions, a solution, etc.) for the computational problem.

In one embodiment, the instruction module 111 may analyze (e.g., automatically analyze) the set of algorithmic operations (e.g., the algorithm, the solution, etc.) to determine whether a set of algorithmic operations can be represented using an algebraic formulation. For example, the set of algorithmic operations may be provided in a specific syntax or format which allows the instruction module 111 to analyze the set of algorithmic operations. The set of algorithmic operations may be received from a user and/or another computing device. For example, a user (e.g., a programmer, engineer, scientist, etc.) may generate and/or provide the set of algorithmic operations using a user interface (e.g., a command line interface, a graphical user interface, etc.).

In one embodiment, the instruction module 111 may generate a set and/or a sequence of idempotent semiring operations based on the set of algorithmic operations in response to determining that the set of algorithmic operations can be represented using the algebraic formulation. For example, if the instruction module 111 determines that the set of algorithmic operations can be represented using the algebraic formulation, the instruction module 111 may generate a set and/or a sequence of idempotent semiring operations based on the set of algorithmic operations and/or the algebraic formulation. A set of idempotent semiring operations may be one or more semiring operations. A sequence of idempotent semiring operations may define which of the idempotent semiring operations may be performed in parallel. For example, a sequence of idempotent semiring operations may indicate an order for the operations (e.g., using parentheses and/or a priority for different operations) and/or the order for the operations may indicate which operations may be performed in parallel.

In one embodiment, the set and/or sequence of idempotent semiring operations (e.g., one or more idempotent semiring operations) may be and/or may represent an algebraic formula. For example, the set and/or sequence of idempotent semiring operations may be an equation and/or formula that includes operands and operations that may be performed on the operands. The operations and/or operations may be in a specific order (e.g., an order of operations). For example, parentheses and/or a priority for different operations may allow the operations to operate on the operands in the specific order. In some embodiments, the instruction module 111 may automatically generate (e.g., determine, obtain, calculate, etc.) the set and/or sequence of idempotent semiring operations based on the set of algorithmic operations (e.g., based on an analysis of the set of algorithmic operations). In other embodiments, the instruction module 111 may optionally receive the set and/or sequence of idempotent semiring operations from a user. For example, the user may provide the set and/or sequence of idempotent semiring operations using a user interface (e.g., a CLI, a GUI, etc.).

In one embodiment, the set and/or sequence of idempotent semiring operations are part of an algebraic semiring. An algebraic semiring may be a type of algebraic structure which consists of a non-empty set, a set/collection of operations on the non-empty set, and a set of identities/axioms that the operations are to satisfy. In particular, an algebraic semiring may be an algebraic structure that lacks the requirement that each element in the semiring must have an additive inverse. In one embodiment, the algebraic semiring (which the set and/or sequence of idempotent semiring operations belong to) may be an algebraic idempotent semiring. An algebraic idempotent semiring may be an algebraic semiring where all elements of the algebraic semiring is an additive idempotent. For example, for each element a in an algebraic idempotent semiring, a+a=a.

In one embodiment, the set and/or sequence of idempotent semiring operations may represent the algebraic formulation (which represents the set of algorithmic operations). For example, the algebraic formulation may be a formula that may represent a solution to a computational problem. The set and/or sequence of idempotent semiring operations may perform the solution to the computational problem.

In one embodiment, the instruction module 111 may generate a set and/or sequence of microcode instructions based on the set and/or sequence of idempotent semiring operations. As discussed above, a set of microcode instructions may include one or more microcode instructions. A sequence of microcode instructions may indicate an order for the instructions and/or may indicate which instructions may be performed in parallel. The set and/or sequence of microcode instructions carry out the set and/or sequence of idempotent semiring operations when the set and/or sequence of microcode instructions is executed by a processing devices (e.g., a systolic array, a processor, etc.) as discuss below. Microcode instructions may be instructions that translate machine code (e.g., machine instructions) into lower layer instructions and/or a binary stream (e.g., a stream of bits).

In one embodiment, the instruction module 111 may modify the set and/or sequence of idempotent semiring operations to reduce the number of operations in the set and/or sequence of idempotent semiring operations. For example, the instruction module 111 may modify the set and/or sequence of idempotent semiring operations by changing the order of the operations to reduce the number of operations. The instruction module 111 may also modify the set and/or sequence of idempotent semiring operations to reduce the amount/number of microcode instructions that are generated.

In one embodiment, the instruction module 111 may receive an indication that a second set and/or sequence of idempotent semiring operations should be used. For example, a user may provide user input (via a user interface) indicating that the second set and/or sequence of idempotent semiring operations should be used. The second set and/or sequence of idempotent semiring operations may represent a second algebraic formulation. The second algebraic formulation may represent a second set of algorithmic operations. The second set and/or sequence of idempotent semiring operations may be part of a second algebraic idempotent semiring. The second algebraic idempotent semiring may include one or more of a second non-empty set, a second set/collection of operations on the second non-empty set, and a second set of identities/axioms that the second set of operations are to satisfy. The instruction module 111 may also generate a second set and/or sequence of microcode instructions based on the second set and/or sequence of idempotent semiring operations.

In one embodiment, each semiring operation in the set of semiring operations may be one or more of an associative commutative pick operation or an associative tally operation. The associative commutative pick operation may form an abelian monoid. For example, for elements a and b, and an operation op, a op b=b op a. The associative commutative pick operation may select a value from a plurality of values (e.g., select a maximum value, a minimum value, etc.). The associative commutative pick operation may be referred to as a pick or a pick operation. The associative tally operation may form a monoid and may distribute over the associate commutative pick operation. For example, for two elements a and b and an operation op, (a op b) op c=a op (b op c). The associate tally operation may generate a generalized product of a set of values. The associative tally operation may be referred to as a tally or a tally operation.

As discussed above, the set of algorithmic operations may be a solution for a computational problem, such as a dynamic programming problem. In one embodiment, the set of algorithmic operations may be a solution for a sequence alignment problem. For example, the set of algorithmic operations may be a solution for how to align two DNA sequences. In another embodiment, the set of algorithmic operations may be a solution for a maximum likelihood decoder. For example, the set of algorithms operations may implement a Viterbi decoder. In a further embodiment, the set of algorithmic operations may be a solution for a shortest path problem. For example, the set of algorithm operations may determine, calculate, generated, a shortest, cheapest, minimum, etc., path between two nodes/vertices in a graph.

In one embodiment, the instruction module 111 may provide the set and/or sequence of microcode instructions to a hardware processing device. The hardware processing device may include a set of processing units configured to receive the set and/or sequence of microcode instructions. The set of processing units are configured for parallelized operations based on one or more of the algebraic formulation and the set and/or sequence of idempotent semiring operations. For example, the set of microinstructions may be executed in parallel in the set of processing units (e.g., each processing unit may execute one instruction of the set of microinstructions in parallel with other processing units).

As illustrated in FIG. 1A, the computing device 120 includes a processing device 126. In one embodiment, the processing device 126 may be a hardware processing device that includes a set of processing units. For example, the processing devices 126 may include a plurality of data processing units (DPUs) that are configured to execute instructions/operations in parallel. The set of processing units may be configured for parallelized operations based on one or more of the algebraic formulation and the set and/or sequence of idempotent semiring operations.

In one embodiment, the processing device 126 may receive the set and/or sequence of microcode instructions generated by the instruction module 111. For example, the instruction module 111 may transmit the microcode instructions to the computing device 120 via the network 105. In another embodiment, the processing device 126 may obtain the microcode instructions from another device. For example, the processing device 126 may read the microcode instructions from a data storage device (e.g., a hard disk drive (HDD), a solid state disk (SSD)) or from a memory.

In one embodiment, the processing device 126 may execute the set and/or sequence of microcode instructions in the set of processing units. For example, the processing device 126 may distribute different microcode instructions to different processing units. Each processing unit may execute a respective set and/or sequence of microcode instructions in parallel with other processing units which are executing their respective sets of microcode instructions.

In one embodiment, the processing device 126 may be capable of performing different operations for different algebraic idempotent semirings. For example, the processing device 126 may be able to perform different sets of idempotent semiring operations for different algebraic idempotent semirings. The processing device 126 and/or the processing units of the processing device 126 (e.g., DPUs of the processing device 126) may be able to switch between different modes or configurations. Each mode/configuration may allow the processing device 126 to perform different operations for the different algebraic idempotent semirings.

In one embodiment, the processing device 126 may receive an indication that a second set and/or sequence of idempotent semiring operations should be used. As discussed above, the second set and/or sequence of idempotent semiring operations may be an equation and/or formula that includes operands and operations that may be performed on the operands. The operations and/or operations may be in a specific order (e.g., an order of operations). The second set and/or sequence of idempotent semiring operations may be part of a second algebraic idempotent semiring. The operations and/or operands in the second set and/or sequence of idempotent semiring operations may be different than the operations and/or operations in the first set and/or sequence of idempotent semiring operations because the first algebraic idempotent semiring may be different than the second algebraic idempotent semiring. The processing device 126 may change to a different configuration/mode than the configuration/mode that was used for the first set and/or sequence of idempotent semiring operations. For example, the processing device 126 may change from a first configuration/mode (for the first set and/or sequence of idempotent semiring operations and/or the first algebraic idempotent semiring) to a second configuration/mode (for the second set and/or sequence of idempotent semiring operations and/or the second algebraic idempotent semiring).

In one embodiment, the processing device 126 may receive a second set and/or sequence of microcode instructions. The second set and/or sequence of microcode instructions may be generated based on the second set and/or sequence of idempotent semiring operations and may be part of a second algebraic idempotent semiring, as discussed above. The processing device 126 may execute the second set and/or sequence of microcode instructions. The processing units of the processing device 126 may further be configured for operations based on one or more of the second algebraic formulation and the second set and/or sequence of idempotent semiring operations (e.g., may be configured to perform different semiring operations, as discussed above).

The processing device 126 may have different architectures in different embodiments. In one embodiment, the processing device 126 may have a single instruction multiple data (SIMD) architecture. A SIMD architecture may be an architecture where the processing device 126 includes multiple processing units/elements that perform the same operation on multiple pieces of data simultaneously. In another embodiment, the processing device 126 may have a single instruction multiple thread (SIMT) architecture. A SIMT architecture may be an architecture where SIMD is combined with multithreading (e.g., where the processing units switch to the same instruction/operation when the processing device 126 changes threads). In a further embodiment, the processing device 126 may have a multiple instruction multiple data (MIMD). A MIMD architecture may be an architecture where the processing device 126 includes multiple processing units/elements that perform the different operations on multiple pieces of data simultaneously.

In one embodiment, the processing device 126 may have an architecture where a processing unit (of the processing device 126) may provide (e.g., broadcast, transmit, send, etc.) a result of an operation to one or more other processing units (e.g., one or more next processing units). For example, the processing device 126 may perform a set of operations (e.g., multiplying two matrices). A processing unit may multiply a first element of a first matrix with a second element of a second matrix. The processing unit may forward the result of the multiplication to one or more other processing units which may add the result with other results. The result that is forwarded to another processing unit (and is used by the other processing unit to perform other operations) may be referred to as a partial result.

In one embodiment, the processing device 126 may be a systolic array. A systolic array may be a network of processing units (e.g., DPUs) which are coupled together. Each processing unit may independently compute a partial result as a function of the data received from a previous (e.g., upstream) processing unit. The partial result computed by a processing unit may be sent downstream to other processing units. A systolic array may be an example of an architecture where processing units provide (e.g., broadcast, transmit, etc.) results to other processing units.

In one embodiment, when the processing device 126 has an architecture where a processing unit (of the processing device 126) may provide a result of an operation to one or more other processing units. Each processing unit may include a memory (e.g., a register, volatile memory, a cache, non-volatile memory, etc.). The memory may store an operand that may be used in an operation performed by the processing unit. The memory may also store a results (e.g., a partial result) of the operation performed by the processing unit.

FIG. 1B is a diagram illustrating an example computing device 130, in accordance with some embodiments of the present disclosure. Computing device 130 may include hardware such as processing device 126, memory (e.g., RAM), storage (HDD, SSD, etc.), and other hardware devices (e.g., sound card, video card, etc.). Computing device 130 may comprise any suitable type of computing device or machine that has a programmable processor including, for example, server computers, desktop computers, laptop computers, tablet computers, smartphones, set-top boxes, etc. In some examples, computing device 130 may comprise a single machine or may include multiple interconnected machines (e.g., multiple servers configured in a cluster).

Computing device 130 includes an instruction module 111. As discussed above, a solution to computational problem may be represented using an algebraic formulation. Instruction module 111 may determine a set and/or sequence of idempotent semiring operations based on the set of algorithmic operations. The instruction module 111 may also generate microcode instructions that may perform the set and/or sequence of idempotent semiring operations when processing device 126 executes the microcode instructions. The idempotent semiring operations may be part of an algebraic semiring.

In one embodiment, the instruction module 111 may determine whether a set of algorithmic operations can be represented using an algebraic formulation. The set of algorithmic operations may be an algorithm (e.g., a set of operations/actions, a solution, etc.) for the computational problem. The set of algorithmic operations may be received from a user and/or another computing device. The instruction module 111 may generate a set and/or sequence of idempotent semiring operations based on the set of algorithmic operations in response to determining that the set of algorithmic operations can be represented using the algebraic formulation. The set of algorithmic operations may be a solution for a computational problem, such as a dynamic programming problem.

As discussed above, the set and/or sequence of idempotent semiring operations (e.g., one or more idempotent semiring operations) may be and/or may represent an algebraic formula (e.g., an equation and/or formula). The instruction module 111 may automatically generate or may receive the set and/or sequence of idempotent semiring operations from a user or other computing device. The set and/or sequence of idempotent semiring operations are part of an algebraic semiring, such as an algebraic idempotent semiring.

In one embodiment, the instruction module 111 may generate a set and/or sequence of microcode instructions based on the set and/or sequence of idempotent semiring operations. The set and/or sequence of microcode instructions carry out the set and/or sequence of idempotent semiring operations when the set and/or sequence of microcode instructions is executed by a processing device 126. The instruction module 111 may optionally modify the set and/or sequence of idempotent semiring operations to reduce the number of operations in the set and/or sequence of idempotent semiring operations.

In one embodiment, the instruction module 111 may receive an indication that a second set and/or sequence of idempotent semiring operations should be used. The second set and/or sequence of idempotent semiring operations may be part of a second algebraic idempotent semiring. The instruction module 111 may also generate a second set and/or sequence of microcode instructions based on the second set and/or sequence of idempotent semiring operations.

In one embodiment, each semiring operation in the set of semiring operations may be one or more of an associative commutative pick operation or an associative tally operation. The associative commutative pick operation (e.g., a pick or a pick operation) may form an abelian monoid. The associative tally operation may form a monoid and may distribute over the associate commutative pick operation. The associative tally operation may be referred to as a tally or a tally operation.

In one embodiment, the instruction module 111 may provide the set and/or sequence of microcode instructions to processing device 126. The processing device 126 may include a set of processing units configured to receive the set and/or sequence of microcode instructions. The set of processing units are configured for parallelized operations based on one or more of the algebraic formulation and the set and/or sequence of idempotent semiring operations. The processing device 126 may receive the set and/or sequence of microcode instructions generated by the instruction module 111. The processing device 126 may also obtain the microcode instructions from another device (e.g., a memory, a SSD).

In one embodiment, the processing device 126 may execute the set and/or sequence of microcode instructions in the set of processing units. For example, the processing device 126 may distribute different microcode instructions to different processing units. Each processing unit may execute a respective set and/or sequence of microcode instructions in parallel with other processing units which are executing their respective sets of microcode instructions.

In one embodiment, the processing device 126 may be capable of performing different operations for different algebraic idempotent semirings. The processing device 126 and/or the processing units of the processing device 126 (may be able to switch between different modes or configurations. Each mode/configuration may allow the processing device 126 to perform different operations for the different algebraic idempotent semirings.

In one embodiment, the processing device 126 may receive an indication that a second set and/or sequence of idempotent semiring operations should be used. The second set and/or sequence of idempotent semiring operations may be part of a second algebraic idempotent semiring. The processing device 126 may change to a different configuration/mode than the configuration/mode that was used for the first set and/or sequence of idempotent semiring operations. In one embodiment, the processing device 126 may receive the second set and/or sequence of microcode instructions and may execute the second set and/or sequence of microcode instructions.

The processing device 126 may have different architectures in different embodiments. For example, the processing device 126 may have a SIMD architecture, a SIMT architecture, or a MIMD architecture. In one embodiment, the processing device 126 may have an architecture where a processing unit (of the processing device 126) may provide (e.g., broadcast, transmit, send, etc.) a result of an operation to one or more other processing units (e.g., one or more next processing units). The result that is forwarded to another processing unit (and is used by the other processing unit to perform other operations) may be referred to as a partial result. In one embodiment, the processing device 126 may be a systolic array. When the processing device 126 has an architecture where a processing unit (of the processing device 126) may provide a result of an operation to one or more other processing units, each processing unit may include a memory. The memory may store an operand that may be used in an operation performed by the processing unit. The memory may also store a results (e.g., a partial result) of the operation performed by the processing unit.

Although the present disclosure may refer to some types of algebraic semirings, other types of algebraic semirings may be used in other embodiments of the present disclosure. Examples of the various algebraic servings that may be used include, but are not limited to a tropical semiring, a k-tropical semiring, a Lukasiewicz semiring, a t-norm semiring, a Viterbi semiring, a matrix semiring, a Boolean semiring, etc. In addition, although the present disclosure may refer to dynamic programming problems, other types of computational problems may be used in other embodiments of the present disclosure. For example, other types of optimization problems may be used.

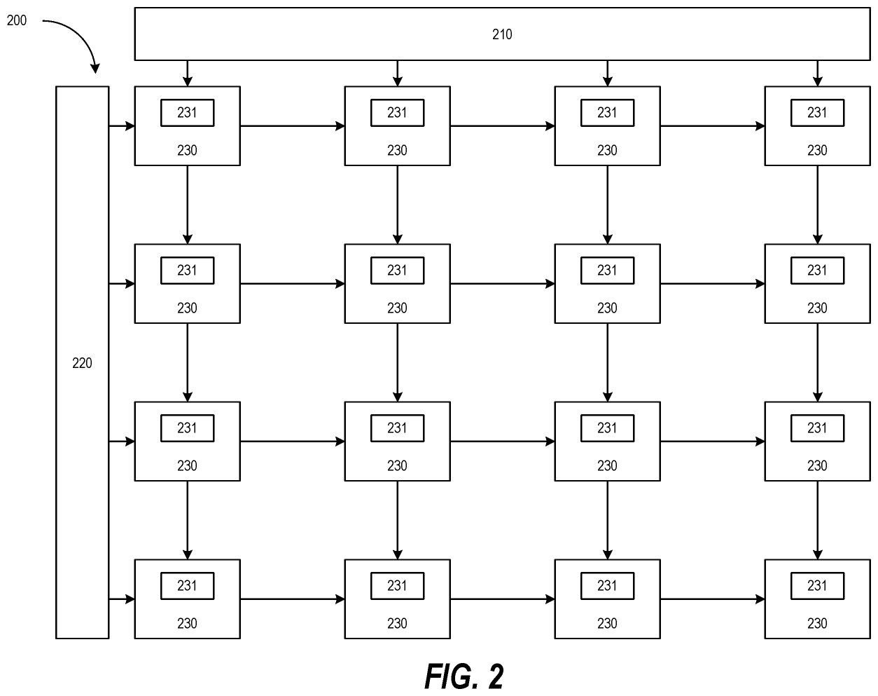

FIG. 2 is a diagram illustrating an example systolic array 200, in accordance with some embodiments of the present disclosure. The systolic array 200 may be an example of a processing device (e.g., processing device 126 illustrated in FIGS. 1A and 1B). Systolic array 200 may be a network of processing units 230. Inputs 210 and 220 are coupled to the systolic array 200. The inputs 210 and 220 may be ports, buses, data lines, wires, pins, cables, etc., where input data is received by the systolic array 200. The top row of processing units 230 is coupled to the input 210. The left most column of processing units is coupled to the input 220. Each processing unit 230 may be coupled to an upstream processing unit 230 (e.g., an upstream processing unit 230) or one of inputs 210 and 230.

In one embodiment, a data processing unit 230 includes a memory 231. The memory may be a register, volatile memory, a cache, non-volatile memory, volatile memory, or some other component (e.g., device, circuit, etc.) that is configured to store data. The memory 231 may store an operand that may be used in an operation performed by the processing unit 230. For example, each memory 231 may store data that was provided to the processing unit 230 as an input (e.g., received from another processing unit 230, received from the input 210 or input 220, etc.). The memory may also store a results (e.g., a partial result) of the operation performed by the processing unit. For example, after the processing unit 230 performs an operation, the result of the operation may be stored in the memory 231.

In one embodiment, each of the data processing units 230 may be identical to each other. For example, each data processing unit 230 may include the same hardware, circuits, memory, input ports/pins, output ports/pins, etc. Each data processing unit 230 may also be capable of performing identical functions/operations. In other embodiments, the data processing units 230 may vary from each other. For example, there may be different sets of data processing units 230 that include different hardware, circuits, memory, etc., and/or that perform different functions/operations.

As illustrated by the arrows in FIG. 2, data (e.g., input data) received from input 210 is provided from one processing unit 230 to another processing unit 230 (e.g., broadcasted from one processing unit 230 to another) in a downward direction. For example, a data processing unit 230 may receive data from a previous data processing unit (e.g., the data processing unit 230 above) and may perform one or more operations on the data. The data processing unit 230 may then provide (e.g., broadcast) the results of the one or more operations to the data processing unit 230 below (e.g., to the downstream or next data processing unit 230).

Also as illustrated by the arrows in FIG. 2, data (e.g., input data) received from input 220 is provided from one processing unit 230 to another processing unit 230 (e.g., broadcasted from one processing unit 230 to another) in a rightward direction. For example, a data processing unit 230 may receive data from a previous data processing unit (e.g., the data processing unit 230 above) and may perform one or more operations on the data. The data processing unit 230 may then provide (e.g., broadcast) the results of the one or more operations to the data processing unit 230 to the right (e.g., to the downstream or next data processing unit 230).

Systolic array 200 may store operands and partial results within the systolic array 200 (e.g., within the memory 231 of the processing units 230). Thus, the systolic array 200 may not access external memory when performing operations, which allows the systolic array 200 to operate more quickly and/or efficiently. In addition, the design of the systolic array 200 makes the systolic array 200 suitable for parallel execution of instructions because each processing unit 230 may operation in parallel. Furthermore, the systolic array 200 may be more efficient when performing operations for a dynamic programming problem because each processing unit 230 operates on a previous partial result and generate a new partial result. This allows the systolic array 200 to perform solutions to dynamic programming problems more quickly and efficiently because each processing unit 230 can perform the operations for one of the sub-problems of the dynamic programming problem. Systolic array 200 may also be useful for artificial intelligence operations, machine learning operations, image processing, pattern recognition, computer vision, etc.

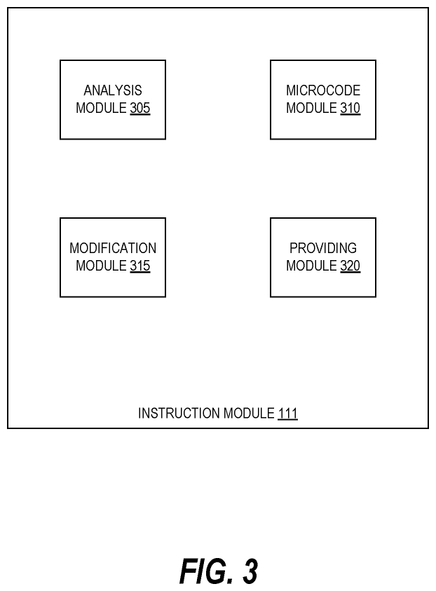

FIG. 3 is a diagram illustrating an example instruction module 111, in accordance with some embodiments of the present disclosure. The instruction module 111 includes, but is not limited to, an analysis module 305, a microcode module 310, a modification module 315, and a providing module 320. Some or all of modules 305-320 may be implemented in software, hardware, or a combination thereof. For example, one or more of modules 305-320 may be installed in persistent storage device, loaded into memory, and executed by one or more processors (not shown). In another example, one or more of modules 305-320 may be hardware, such as circuits, processing devices, such as an application specific integrated circuit (ASIC), a field programmable gate array (FPGA), etc. Some of modules 305-320 may be integrated together as an integrated module. In addition, some of modules 305-320 may be located in difference computing devices (e.g., different server computers). In some embodiments, the instruction module 111 may be referred to as a compiler.

In one embodiment, the analysis module 305 may determine whether a set of algorithmic operations can be represented using an algebraic formulation. The set of algorithmic operations may be an algorithm for the computational problem. The set of algorithmic operations may be received from a user and/or another computing device (e.g., may be included in an input file, received via a user interface, etc.). The analysis module 305 may generate a set and/or sequence of idempotent semiring operations based on the set of algorithmic operations in response to determining that the set of algorithmic operations can be represented using the algebraic formulation.

In one embodiment, the microcode module 310 may generate a set and/or sequence of microcode instructions based on the set and/or sequence of idempotent semiring operations. The set and/or sequence of microcode instructions carry out the set and/or sequence of idempotent semiring operations when the set and/or sequence of microcode instructions is executed by a processing device.

In one embodiment, the modification module 315 may modify the set and/or sequence of idempotent semiring operations to reduce the number of operations in the set and/or sequence of idempotent semiring operations and/or to reduce the amount/number of microcode instructions that are generated.

In one embodiment, the providing module 320 may provide the set and/or sequence of microcode instructions to a processing device. For example, the providing module 320 may transmit the set and/or sequence of microcode instructions to the processing device via a bus, network, etc. The processing device may include a set of processing units configured to receive the set and/or sequence of microcode instructions. The set of processing units are configured for parallelized operations based on one or more of the algebraic formulation and the set and/or sequence of idempotent semiring operations.

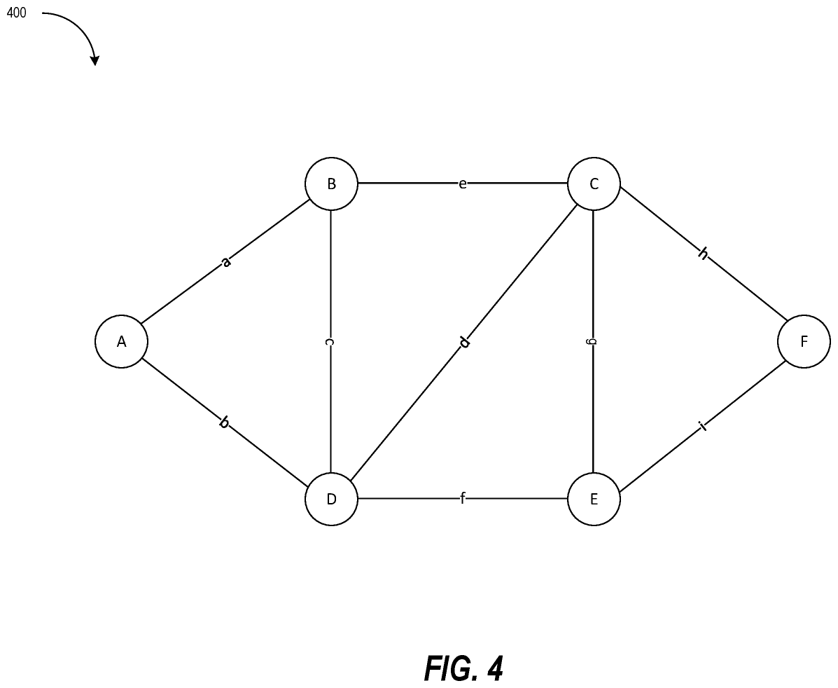

FIGS. 4-7 illustrate various example applications of transforming or decomposing dynamic programming algorithms/problems into formulation of sequence of idempotent semiring operations. As discussed above, those operations within the formulation can readily lead to optimized microcode instructions that can be sent to a processing device for efficient and parallelized execution.

FIG. 4 is a diagram illustrating an example graph 400 associated with an example algorithm to illustrate representation using an algebraic formulation, in accordance with some embodiments of the present disclosure. The graph 400 includes nodes (e.g., vertices) A, B, C, D, E, and F. The nodes A, B, C, D, E, and F are connected via edges. Each of the edges may be associated with a cost for using the respective edge to traverse the graph 400. The cost of an edge is representing using a, b, c, d, e, f, g, h, and i. For example, going from node A to node B may incur a cost of a, going from node C to node D may incur a cost of d, going from node E to node F may incur a cost of i, etc. Thus, graph 400 may be referred to as a weighted graph.

Various algorithms may be used to determine the shortest (e.g., optimal, lowest cost, etc.) path from A to F. These algorithms may be referred to as shortest path algorithms. One such algorithm may be the Floyd-Warshall algorithm. The Floyd-Warshall algorithm may be represented with the following equation:

shortestPath(i,j,k)=min(shortestPath(i,j,k−1),(shortestPath(i,k,k−1)+shortestPath(k,j,k−1))) (1)

i is the starting point, j is the destination, and k is the set of nodes/vertices with the weighted graph 400.

In one embodiment, the formula (1) above may be represented using an algebraic formulation. For example, an instruction module (e.g., instruction module 111 illustrated in FIGS. 1A and 1B) may determine the following algebraic formulation for formula (1):

F°=(C°⊗h)(E°⊗i) (2a)

The lowest cost to reach a node X from node A may be represented as X°. For example, the lowest cost to reach node C from node A is represented as C°. The term C° in equation (2a) can be defined as follows:

C°=B°⊗e⊕D°⊗d⊕E°⊗g (2b)

And the term E° in equation (2a) can be defined as follows:

E°=D°⊗f⊕C°⊗g (2c)

Each cost term (e.g., X°) in each of equations (2b) and (2c) may be defined using additional equations until we reach the starting point A. The additional equations are not shown here.

The ⊕ operation may be referred to as a commutative pick operation or a pick operation. The ⊕ operation may indicate the best value/choice (e.g., the lowest cost) between two operations. For example, X⊕Y may indicate that the lowest of X or Y should be selected. The ⊗ operation may indicate that that the best values/choices (e.g., the lowest costs paths) that were selected earlier should be tallied (e.g., summed, added together, etc.). The ⊗ operation may be referred to as a tally operation or an associate tally operation.

In one embodiment, formulas (2c-2c) may be a set and/or sequence of idempotent semiring operations that are part of an algebraic semiring, such as an algebraic idempotent semiring. The ⊕ operation may also be referred to as a generalized addition. The ⊕ operation may also satisfy the following properties: 1) (A⊕B)⊕C=A⊕(B⊕C); 2) A⊕B=B⊕A; and 3) 0⊕A=A⊕0. Thus, the ⊕ operation may form an abelian monoid. The ⊗ operation may also be referred to as a generalized multiplication. The ⊗ operation may also satisfy the following properties: 1) (A⊗B)⊗C=A⊗(B⊗C); but in the general case 2) A⊗B!=B⊗A. Thus, the ⊗ operation may form a monoid. Together, the ⊕ operation and the ⊗ operation form an algebraic semiring. In particular, the ⊕ operation and the ⊗ operation form a tropical semiring, which may also be referred to as (min, +) algebra.

As discussed above, representing a computational problem (e.g., the solution to a computational problem) using idempotent semiring operations (which are part of or which form an algebraic semiring) may be useful. For example, the embodiments described herein allow the solution to a computational program to be defined using an algebraic representation. The use of idempotent semiring operations allows the dynamic programming problem to be represented using an algebraic formulation which may be bounded by a limited set of operations under a sequence of operations (e.g., bounded with operators that have pre-defined properties). This decomposition into a formalistic expression enables ease of hardware efficiency tuning and parallelized execution. Efficiency can be gained also due to the limited number of idempotent semiring operations involved and hardware can be discretized or otherwise optimized for those operations. In addition, knowledge about how the underlying hardware will execute instructions may not be needed because the microcode instructions are generated such that they are easy to execute in parallel, since the order or sequence of operations in the formulation (along with specific properties (e.g., communicative) related to the operators) define what operations can be done in parallel and what operations need to follow an order or sequence. This may allow programs, applications, etc., to be created, generated, written, etc., more easily. This may also allow for a higher degree of parallelism in the operation/execution of a program, application, etc.

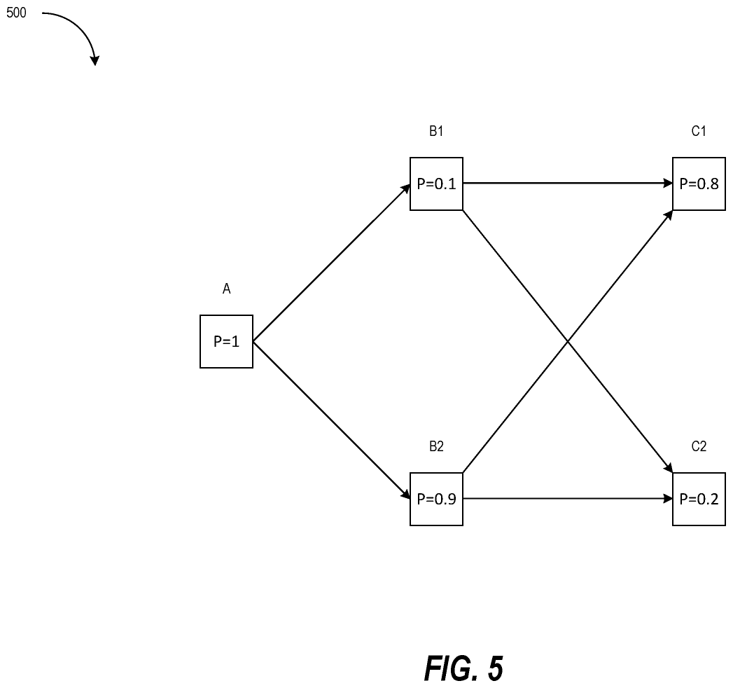

FIG. 5 is a diagram illustrating an example decoder 500 for decoding a bit stream, in accordance with some embodiments of the present disclosure. In one embodiment, the decoder 500 may be a maximum likelihood decoder, such as a Viterbi decoder, which may be used in data storage and data communication applications for recovery of data from a storage or transmission medium. A Viterbi decoder may decode bitstreams (e.g., a sequence of bits, a symbol, etc.) that are encoded using a convolutional code. As illustrated in FIG. 5, the Viterbi decode may be represented using a graph of connected nodes A, B1, B2, C1, and C2. Each of the nodes A, B1, B2, C1, and C2 is associated with a symbol that may part of the decoded bitstream. The cost for reaching each node is indicated by P=x where x is the cost. The nodes that are part of cheapest path (e.g., the path with the lowest/smallest cost) are the symbols that will be generated by the decoder 500 when the decoder 500 decodes the bitstream.

In one embodiment, determining or identifying the lowest cost path for the graph (which may indicate how a bitstream will be decoded by the decoder 500) can be represented using the following formula:

c(path)=min(c(C1)+min(c(B1)+c(A),c(B2)+c(A)),c(C2)+min(c(B1)+c(A),c(B2)+c(A))). (3)

The min( ) function selects the minimum value of values/parameters provided to the min( ) function. For example, min (X, Y) selects the minimum value between X and Y. The cost function c( ) determines the cost for getting to one of the nodes B1, B2, C1, and C1 from node A. For example, c(B1) represent the cost of betting to B1 from A.

In one embodiment, the formula (1) above may be represented using an algebraic formulation. For example, an instruction module (e.g., instruction module 111 illustrated in FIGS. 1A and 1B) may determine the following algebraic formulation for formula (3):

c(path)=c(C1)⊗c(B1)⊗c(A)⊕c(B2)⊗c(A)⊕c(C2)⊗c(B1)⊗c(A)⊕c(B2)⊗c(A) (4)

The min( ) function of formula (3) is represented using ⊕ operation. For example, min (X, Y) may be represented using X⊕Y. The ⊕ operation may be referred to as a commutative pick operation or a pick operation. The + function of formula (3) is represented using the ⊗ operation. For example, X+Y may be represented using X⊗Y. The ⊗ may indicate that the best values/choices (e.g., the lowest costs paths) that were selected earlier should be tallied (e.g., summed, added together, etc.). The ⊗ operation may be referred to as a tally operation or an associate tally operation.

In one embodiment, formula (4) may be a set and/or sequence of idempotent semiring operations that are part of an algebraic semiring, such as an algebraic idempotent semiring. The ⊕ operation may also be referred to as a generalized addition. The ⊕ operation may also satisfy the following properties: 1) (A⊕B)⊕C=A⊕(B⊕C); 2) A⊕B=B⊕A; and 3) 0⊕A=A⊕0. Thus, the ⊕ operation may form an abelian monoid. The ⊗ operation may also be referred to as a generalized multiplication. The ⊗ operation may also satisfy the following properties: 1) (A⊗B)⊗C=A⊗(B⊗C); and 2) 1⊗A=A⊗1. Thus, the ⊗ operation may form a monoid. Together, the ⊕ operation and the ⊗ operation form an algebraic semiring. In particular, the ⊕ operation and the ⊗ operation form a tropical semiring, which may also be referred to as (min, +) algebra.

As discussed above, representing a computational problem (e.g., the solution to a computational problem) using idempotent semiring operations (which are part of or which form an algebraic semiring) may be useful. For example, the embodiments described herein allow the solution to a computational program to be defined using an algebraic representation. The use of idempotent semiring operations allows the dynamic programming problem to be represented using an algebraic formulation which may be bounded by a limited set of operations under a sequence of operations (e.g., bounded with operators that have pre-defined properties). This decomposition into a formalistic expression enables ease of hardware efficiency tuning and parallelized execution. Efficiency can be gained also due to the limited number of idempotent semiring operations involved and hardware can be discretized or otherwise optimized for those operations. In addition, knowledge about how the underlying hardware will execute instructions may not be needed because the microcode instructions are generated such that they are easy to execute in parallel, since the order or sequence of operations in the formulation (along with specific properties (e.g., communicative) related to the operators) define what operations can be done in parallel and what operations need to follow an order or sequence. This may allow programs, applications, etc., to be created, generated, written, etc., more easily. This may also allow for a higher degree of parallelism in the operation/execution of a program, application, etc.

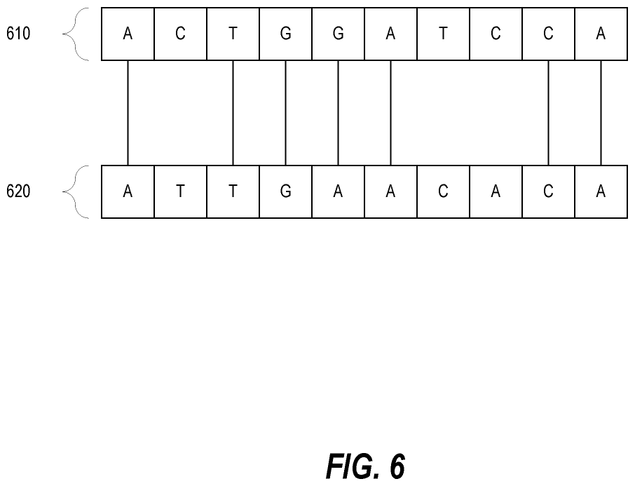

FIG. 6 is a diagram illustration example DNA sequences 610 and 620, in accordance with some embodiments of the present disclosure. A DNA sequence may be string, sequence, etc., that consist of the bases represented by the letters “A,” “T,” “C,” and “G.” Each of the letters represent a nucleotide that may be part of a DNA sequence. The letter “A” represents the nucleotide adenine. The letter “T” represents the nucleotide thymine. The letter “C” represents the nucleotide cytosine. The letter “G” represents the nucleotide guanine.

In the field of bioinformatics, identifying alignments between different sequences of DNA is an important and useful operation. Two DNA sequences may be aligned when a threshold number of letters (e.g., elements) in the DNA sequence match based on their positions, as discussed in more detail below. The process of identifying alignments between different sequences of DNA may be referred to finding or identifying a sequence alignment. Identifying a sequence alignment (e.g., an alignment of two DNA sequences) may allow for identification of regions of similarity between different DNA sequences. These regions of similarity may allow users to predict the function of a DNA sequence and/or may allow users to find specific genes of genomes.

As illustrated in FIG. 6, DNA sequences 610 and 620 each include ten letters (bases) in ten different positions. The first, third, fourth, fifth, sixth, ninth, and tenth positions have matching letters between the DNA sequences 610 and 620 (as indicated by the line between the two DNA sequences 610 and 620). Various algorithms may be used to perform a sequence alignment between two DNA sequences. One such algorithm is the Smith-Waterman algorithm. The Smith-Waterman algorithm generates a scoring matrix and traces back through the scoring matrix to determine how to best align two DNA sequences.

The Smith-Waterman algorithm may operate as follows. Let A=α₁α₂ . . . aₙ and B=b₁b₂ . . . bₘ be the sequences to be aligned, where n and m are the lengths of A and B respectively. A scoring matrix H is constructed, the size of the scoring matrix is (n+1)*(m+1). The scoring matrix H is populated (e.g. filled) as follows:

$${H}_{ij} = max \{ \begin{matrix}{H}_{i - 1 , j - 1} + s ( {a}_{i} , {b}_{j} ) , \\ {max}_{k \geq 1} \{ {H}_{i - k , j} - {W}_{k} \} , \\ {max}_{l \geq 1} \{ {H}_{i , j - l} - {W}_{l} \} , \\ 0\end{matrix} ( 1 \leq i \leq n , 1 \leq j \leq m )$$

where H_{i-1,j-1}+s(aᵢ,bⱼ) is the score of aligning aᵢ and bⱼ;
where H_{i-k,j}−Wₖ is the score if a, is at the end of a gap of length k;
where H_{i,j-1}−Wₗ is the score if bⱼ is at the end of a gap of length l; and
where 0 means there is no similarity up to aᵢ and bⱼ.

In one embodiment, the Smith-Waterman algorithm (shown above) may be represented using an algebraic formulation. For example, an instruction module (e.g., instruction module 111 illustrated in FIGS. 1A and 1B) may determine an algebraic formulation for calculating the different elements of the scoring matrix. Example 1 in Appendix A may provide an example of how the calculation of the scoring matrix may be represented using an algebraic formulation which is represented using a set and/or sequence of idempotent semiring operations that are part of an algebraic semiring, such as an algebraic idempotent semiring. In example 1 (of Appendix A), there are two DNA sequences A=a₁a₂a₃a₄ and B=b₁b₂b₃b₄ (e.g., each DNA sequence has a length of 4). To determine an alignment for the two DNA sequences A and B, a scoring matrix is constructed as follows:

[[00 0 0 0]
[0 H(1, 1) H(1, 2) H(1, 3) H(1, 4)]
[0 H(2, 1) H(2, 2) H(2, 3) H(2, 4)]
[0 H(3, 1) H(3, 2) H(3, 3) H(3, 4)]
[0 H(4, 1) H(4, 2) H(4, 3) H(4, 4)]]
   Each of the functions H(X, Y) may be calculated based on the idempotent operations indicated in Example 1 of Appendix A. For example, H(1,1)=0⊗s(b0, a0), H(1,2)=W1⊗(0⊗s(b0, a0)) ⊕ 0⊗s(b0, a1), etc.

In one embodiment, the idempotent semiring operations indicated in Example 1 of Appendix A are part of an algebraic semiring, such as an algebraic idempotent semiring. The ⊕ operation may also be referred to as a generalized addition. The ⊕ operation may also satisfy the following properties: 1) (A⊕B)⊕C=A⊕(B⊕C); 2) A⊕B=B⊕A; and 3) 0⊕A=A⊕0. Thus, the ⊕ operation may form an abelian monoid. The ⊗ operation may also be referred to as a generalized multiplication. The ⊗ operation may also satisfy the following properties: 1) (A⊗B)⊗C=A⊗(B⊗C); and 2) 1⊗A=A⊗1. Thus, the ⊗ operation may form a monoid. Together, the ⊕ operation and the ⊗ operation form an algebraic semiring. In particular, the ⊕ operation and the ⊗ operation form a tropical semiring.

As discussed above, representing a computational problem (e.g., the solution to a computational problem) using idempotent semiring operations (which are part of or which form an algebraic semiring) may be useful. For example, the embodiments described herein allow the solution to a computational program to be defined using an algebraic representation. The use of idempotent semiring operations allows the dynamic programming problem to be represented using an algebraic formulation which may be bounded by a limited set of operations under a sequence of operations (e.g., bounded with operators that have pre-defined properties). This decomposition into a formalistic expression enables ease of hardware efficiency tuning and parallelized execution. Efficiency can be gained also due to the limited number of idempotent semiring operations involved and hardware can be discretized or otherwise optimized for those operations. In addition, knowledge about how the underlying hardware will execute instructions may not be needed because the microcode instructions are generated such that they are easy to execute in parallel, since the order or sequence of operations in the formulation (along with specific properties (e.g., communicative) related to the operators) define what operations can be done in parallel and what operations need to follow an order or sequence. This may allow programs, applications, etc., to be created, generated, written, etc., more easily. This may also allow for a higher degree of parallelism in the operation/execution of a program, application, etc.

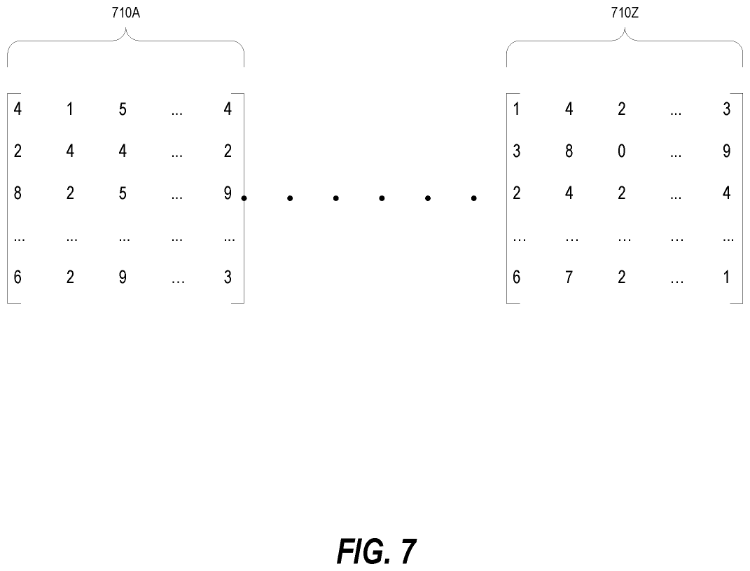

FIG. 7 is a diagram illustrating example matrices 710A through 710Z, in accordance with some embodiments of the present disclosure. As discussed above, matrices may be used in algorithmic operations that may represent a solution to a computational problem. For example, a scoring matrix is used in DNA sequence alignment. As illustrated in FIG. 7, multiple matrices 710A through 710Z may be multiplied with each other when performing a set and/or sequence of idempotent semiring operations. Because matrix operations (e.g., matric multiplication) involves multiple the numbers in the rows of numbers of a first matrix with the numbers in the columns of a second matrix, the number of operations (e.g., the number of multiplications and additions) may increase as you multiple more and more matrices.

Due to the large number of operations when multiplying matrices, it may be important to optimize the order of the matrix multiplications to reduce the number of operations that are performed (e.g., to reduce the number of multiplications/additions, which reduces the number of idempotent semiring operations which may further reduce the number or microcode instructions that are generated. For example, if there are three matrices A, B, and C, and A is a 10×30 matrix, B is a 30×5 matrix, and C is a 5×60 matrix, then computing A(BC) uses (30×5×60)+(10×30×60)=9000+18000=27000 operations. However, changing the order of the operations and computing (AB)C uses (10×30×5)+(10×5×60)=1500+3000=4500 operations. Determining the optimal order for multiplying matrices may be referred to as a matrix chain ordering problem (MCOP). In some embodiments, an instruction module may analyze the set and/or sequence of idempotent semiring operations and/or the set of algorithmic operations to identify the optimal order for multiplying matrices. Various algorithms, techniques, and/or methods may be used to identify the optimal order for multiplying matrices.

In other embodiments, the instruction module may use vectors and/or tensors in the idempotent semiring operations. For example, some computational problems may use many-to-one or many-to-many operations (e.g., vector and/or matrix operations). The instruction module may use pick and tally operations (e.g., ⊕ and ⊗ operations) which operator on vectors and/or tensors. For example, the instruction module may generate operations that use vectors/tensors as inputs and/or output vectors/tensors. By using vector/tensor operations, the instruction module may be able to achieve a high level of data parallelism and/or may be able to achieve more efficient execution. For example, by generating vector/tensor operations which may be distributed across multiple processing units of a processing device, the instruction module allows a higher level of data parallelism and/or more efficient execution.

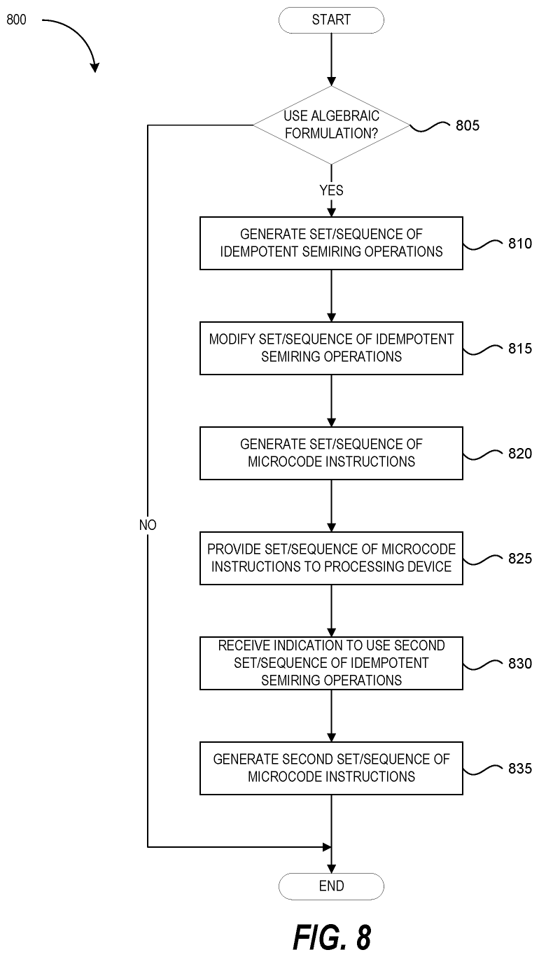

FIG. 8 is a flowchart illustrating an example a process 800 for generating microcode instructions, in accordance with one or more embodiments of the present disclosure. The process 800 may be performed by a processing device (e.g., a processor, a central processing unit (CPU), a graphical processing units (GPU), a controller, an application-specific integrated circuit (ASIC), a field programmable gate array (FPGA), etc.) and/or an instruction module. For example, the process 800 may be performed by a processing device of a sensor device (e.g., a secondary sensor device). The processing device and/or instruction module may be processing logic that includes hardware (e.g., circuitry, dedicated logic, programmable logic, microcode, etc.), software (e.g., instructions run on a processor to perform hardware simulation), firmware, or a combination thereof.

The process 800 begins at block 805 where the process 800 determines whether a set of algorithmic operations can be represented using an algebraic formulation. For example, the process 800 may analyze data, metadata, an input file, etc., that includes the set of algorithmic operations in a syntax/format. As discussed above, the set of algorithmic operations may be a solution to a computational problem, such as a dynamic programming problem. If the set of algorithmic operations cannot be represented using an algebraic formulation, the process 800 ends.

If a set or part of a set of algorithmic operations can be represented using an algebraic formulation, the process 800 proceeds to block 810, where the process 800 generates a set and/or sequence of idempotent semiring operations. As discussed above, the set and/or sequence of idempotent semiring operations are part of an algebraic idempotent semiring. The set and/or sequence of idempotent semiring operations may also represent the algebraic formulation. At block 815, the process 800 may optionally modify the set and/or sequence of idempotent semiring operations. For example, the process 800 may change the order of some of the idempotent semiring operations. At block 820, the process 800 may generate a set and/or sequence of microcode instructions based on the set and/or sequence of idempotent semiring operations. The set and/or sequence of microcode instructions carry out the set and/or sequence of idempotent semiring operations. At block 825, the process 800 may optionally provide the set and/or sequence of microcode instructions to a processing device. For example, the process 800 may transmit the set and/or sequence of microcode instructions to the processing device. As discussed above, the processing device may include a set of processing units configured to receive the set and/or sequence of microcode instructions. The set of processing units may also be configured for parallelized operations based on one or more of the algebraic formulation and the set and/or sequence of idempotent semiring operations.

At block 830, the process 800 may optionally receive an indication to use a second set and/or sequence of idempotent semiring operations. For example, the process may receive an indication that a second algebraic idempotent semiring should be used and the process 800 may generate the second set and/or sequence of idempotent semiring operations which may be part of the second algebraic idempotent semiring. At block 835, the process 800 may optionally generate a second set and/or sequence of microcode instructions based on the set and/or sequence of idempotent semiring operations.

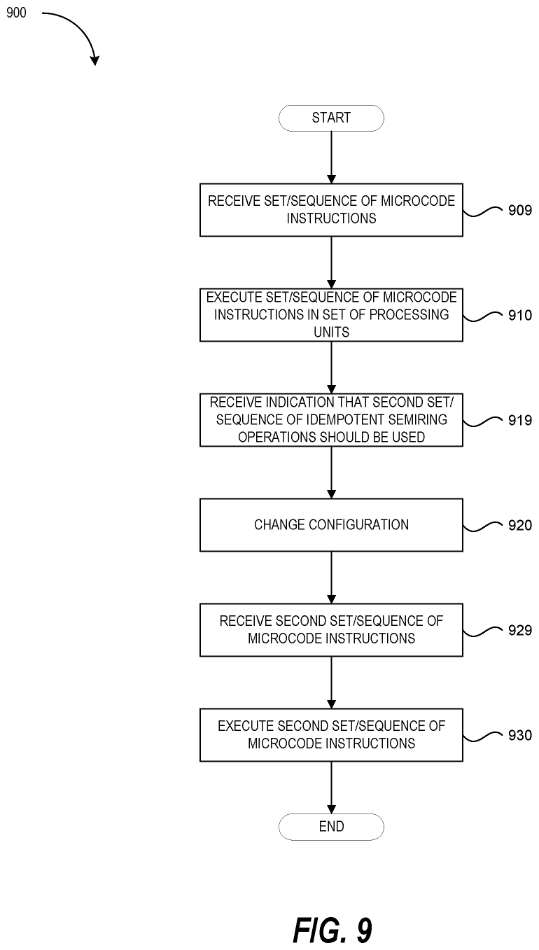

FIG. 9 is a flowchart illustrating an example a process 900 for generating microcode instructions, in accordance with one or more embodiments of the present disclosure. The process 900 may be performed by a processing device (e.g., a processor, a central processing unit (CPU), a graphical processing units (GPU), a controller, an application-specific integrated circuit (ASIC), a systolic array, a field programmable gate array (FPGA), etc.) and/or an instruction module. For example, the process 900 may be performed by a processing device as illustrated in FIGS. 1A and 1B. The processing device and/or instruction module may be processing logic that includes hardware (e.g., circuitry, dedicated logic, programmable logic, microcode, etc.), software (e.g., instructions run on a processor to perform hardware simulation), firmware, or a combination thereof.

The process 900 begins at block 905 where the process 900 receives a set and/or sequence of microcode instructions. The set and/or sequence of microcode instructions may be generated by an instruction module, as discussed above. The set and/or sequence of microcode instructions may be based on a set and/or sequence of idempotent semiring operations. The set and/or sequence of idempotent semiring operations may be part of an algebraic idempotent semiring. The set and/or sequence of idempotent semiring operations may represent an algebraic formulation representing a set of algorithmic operations. At block 905, the process 900 may execute the set and/or sequence of microcode instructions in a set of processing units (e.g., DPUs) of the processing device. The set and/or sequence of microcode instructions carry out the set and/or sequence of idempotent semiring operations. The set of processing units may be configured for parallelized operations based on one or more of the algebraic formulation and the set and/or sequence of idempotent semiring operations.

At block 915, the process 900 may optionally receive an indication that a second set and/or sequence of idempotent semiring operations should be used. The second set and/or sequence of idempotent semiring operations which may be part of the second algebraic idempotent semiring. At block 920, the process 900 may optionally change a configuration, mode, etc., of the processing device and/or processing units. The new mode/configuration may allow the processing device and/or processing units to perform idempotent semiring operations for the second algebraic idempotent semiring. At block 950, the process 900 may optionally execute the second set and/or sequence of microcode instructions.

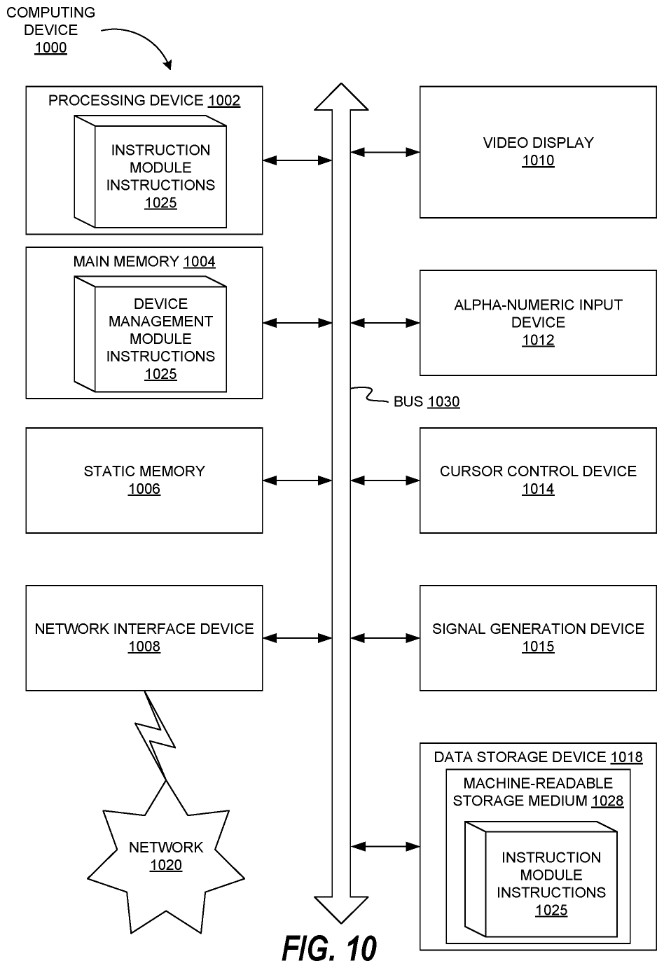

FIG. 10 is a block diagram of an example computing device 1000 that may perform one or more of the operations described herein, in accordance with some embodiments. Computing device 1000 may be connected to other computing devices in a LAN, an intranet, an extranet, and/or the Internet. The computing device may operate in the capacity of a server machine in client-server network environment or in the capacity of a client in a peer-to-peer network environment. The computing device may be provided by a personal computer (PC), a set-top box (STB), a server, a network router, switch or bridge, or any machine capable of executing a set of instructions (sequential or otherwise) that specify actions to be taken by that machine. Further, while only a single computing device is illustrated, the term “computing device” shall also be taken to include any collection of computing devices that individually or jointly execute a set (or multiple sets) of instructions to perform the methods discussed herein.

The example computing device 1000 may include a processing device (e.g., a general purpose processor, a programmable logic device (PLD), etc.) 1002, a main memory 1004 (e.g., synchronous dynamic random access memory (DRAM), read-only memory (ROM)), a static memory 1006 (e.g., flash memory), and a data storage device 1018), which may communicate with each other via a bus 1030.

Processing device 1002 may be provided by one or more general-purpose processing devices such as a microprocessor, central processing unit, or the like. In an illustrative example, processing device 1002 may comprise a complex instruction set computing (CISC) microprocessor, reduced instruction set computing (RISC) microprocessor, very long instruction word (VLIW) microprocessor, or a processor implementing other instruction sets or processors implementing a combination of instruction sets. Processing device 1002 may also comprise one or more special-purpose processing devices such as an application specific integrated circuit (ASIC), a field programmable gate array (FPGA), a digital signal processor (DSP), network processor, or the like. The processing device 1002 may be configured to execute the operations described herein, in accordance with one or more aspects of the present disclosure, for performing the operations and steps discussed herein.

Computing device 1000 may further include a network interface device 1008 which may communicate with a network 1020. The computing device 1000 also may include a video display unit 1010 (e.g., a liquid crystal display (LCD) or a cathode ray tube (CRT)), an alphanumeric input device 1012 (e.g., a keyboard), a cursor control device 1014 (e.g., a mouse) and an acoustic signal generation device 1016 (e.g., a speaker). In one embodiment, video display unit 1010, alphanumeric input device 1012, and cursor control device 1014 may be combined into a single component or device (e.g., an LCD touch screen).

Data storage device 1018 may include a computer-readable storage medium 1028 on which may be stored one or more sets of instruction module instructions 1025, e.g., instructions for carrying out the operations described herein, in accordance with one or more aspects of the present disclosure. Instruction module instructions 1025 may also reside, completely or at least partially, within main memory 1004 and/or within processing device 1002 during execution thereof by computing device 1000, main memory 1004 and processing device 1002 also constituting computer-readable media. The instruction module instructions 1025 may further be transmitted or received over a network 1020 via network interface device 1008.

While computer-readable storage medium 1028 is shown in an illustrative example to be a single medium, the term “computer-readable storage medium” should be taken to include a single medium or multiple media (e.g., a centralized or distributed database and/or associated caches and servers) that store the one or more sets of instructions. The term “computer-readable storage medium” shall also be taken to include any medium that is capable of storing, encoding or carrying a set of instructions for execution by the machine and that cause the machine to perform the methods described herein. The term “computer-readable storage medium” shall accordingly be taken to include, but not be limited to, solid-state memories, optical media and magnetic media.

### General Comments

Those skilled in the art will appreciate that in some embodiments, other types of distributed data storage systems may be implemented while remaining within the scope of the present disclosure. In addition, the actual steps taken in the processes discussed herein may differ from those described or shown in the figures. Depending on the embodiment, certain of the steps described above may be removed, others may be added.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. The accompanying claims and their equivalents are intended to cover such forms or modifications as would fall within the scope and spirit of the protection. For example, the various components illustrated in the figures may be implemented as software and/or firmware on a processor, ASIC/FPGA, or dedicated hardware. Also, the features and attributes of the specific embodiments disclosed above may be combined in different ways to form additional embodiments, all of which fall within the scope of the present disclosure. Although the present disclosure provides certain preferred embodiments and applications, other embodiments that are apparent to those of ordinary skill in the art, including embodiments which do not provide all of the features and advantages set forth herein, are also within the scope of this disclosure. Accordingly, the scope of the present disclosure is intended to be defined only by reference to the appended claims.

The words “example” or “exemplary” are used herein to mean serving as an example, instance, or illustration. Any aspect or design described herein as “example’ or “exemplary” is not necessarily to be construed as preferred or advantageous over other aspects or designs. Rather, use of the words “example” or “exemplary” is intended to present concepts in a concrete fashion. As used in this disclosure, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this disclosure and the appended claims should generally be construed to mean “one or more” unless specified otherwise or clear from context to be directed to a singular form. Moreover, use of the term “an embodiment” or “one embodiment” or “an implementation” or “one implementation” throughout is not intended to mean the same embodiment or implementation unless described as such. Furthermore, the terms “first,” “second,” “third,” “fourth,” etc., as used herein are meant as labels to distinguish among different elements and may not necessarily have an ordinal meaning according to their numerical designation.

All of the processes described above may be embodied in, and fully automated via, software code modules executed by one or more general purpose or special purpose computers or processors. The code modules may be stored on any type of computer-readable medium or other computer storage device or collection of storage devices. Some or all of the methods may alternatively be embodied in specialized computer hardware.

What is claimed is:

1. An apparatus, comprising:
a memory configured to store a sequence of microcode instructions, wherein:
a subset of the sequence of microcode instructions are based on a set of idempotent semiring operations;
the set of idempotent semiring operations are part of an algebraic idempotent semiring; and
the set of idempotent semiring operations represent an algebraic formulation representing a set of algorithmic operations;
a hardware processing device operatively coupled to the memory and comprising a set of processing units configured to:
receive the sequence of microcode instructions, wherein:
the sequence of microcode instructions carries out the set of idempotent semiring operations; and
the set of processing units are configured for parallelized operations based on one or more of the algebraic formulation and the set of idempotent semiring operations;
and
execute the sequence of microcode instructions in the set of processing units.

2. The apparatus of claim 1, wherein the sequence of microcode instructions are executed in parallel in the set of processing units.

3. The apparatus of claim 1, wherein the hardware processing device is further configured to:
receive an indication that a second set of idempotent semiring operations should be used, wherein:
the second set of idempotent semiring operations represent a second algebraic formulation; and
the second set of idempotent semiring operations are part of a second algebraic idempotent semiring;
receive a second sequence of microcode instructions, wherein the second sequence of microcode instructions are generated based on the second set of idempotent semiring operations; and
execute the second sequence of microcode instructions, wherein the set of processing units are further configured for operations based on one or more of the second algebraic formulation and the second set of idempotent semiring operations.

4. The apparatus of claim 1, wherein:
each of the set of idempotent semiring operations comprises one or more of an associative, commutative pick operation that forms an abelian monoid and an associative tally operation that forms a monoid and distributes over the pick operation;
the associate commutative pick operation selects a value from a first plurality of values; and
the associative tally operation generates a generalized product of a second plurality of values.

5. The apparatus of claim 1, wherein the hardware processing device comprises a systolic array and wherein the set of processing units comprises a set of data processing units.

6. The apparatus of claim 1, wherein the hardware processing device comprises a single instruction multiple thread (SIMT) architecture.

7. The apparatus of claim 1, wherein the hardware processing device comprises a single instruction multiple data (SIMD) architecture.

8. The apparatus of claim 1, wherein the hardware processing device comprises a multiple instruction multiple data (MIMD) architecture.

9. The apparatus of claim 1, wherein each processing unit of the set of processing units provides a partial result to a respective next processing unit.

10. The apparatus of claim 8, wherein each processing unit of the set of processing units comprises a memory configured to store an operand.

11. The apparatus of claim 1, wherein the set of algorithmic operations comprise operations for determining a solution for a dynamic programming problem.

12. The apparatus of claim 11, wherein the set of algorithmic operations comprise operations for determining a solution for aligning nucleotide sequences.

13. The apparatus of claim 11, wherein the set of algorithmic operations comprise operations for determining a solution for a maximum likelihood decoder.

14. The apparatus of claim 1, wherein the algebraic idempotent semiring comprises one or more of: a tropical semiring, a k-tropical semiring, a Lukasiewicz semiring, a t-norm semiring, a Viterbi semiring, a matrix semiring, and a Boolean semiring.

15. A method, comprising:
obtaining a sequence of microcode instructions, wherein:
a subset of the sequence of microcode instructions are based on a set of idempotent semiring operations;
the set of idempotent semiring operations are part of an algebraic idempotent semiring;
the set of idempotent semiring operations comprise one or more of an associative, commutative pick operation that forms an abelian monoid and an associative tally operation that forms a monoid and distributes over the pick operation;
the set of idempotent semiring operations represent an algebraic formulation representing a set of algorithmic operations; and
the sequence of microcode instructions carries out the set of idempotent semiring operations; and
executing the sequence of microcode instructions in a set of processing units of a hardware processing device, wherein the set of processing units are configured for parallelized operations based on one or more of the algebraic formulation and the set of idempotent semiring operations.

16. The method of claim 15, further comprising:
receiving an indication that a second set of idempotent semiring operations should be used, wherein:
the second set of idempotent semiring operations represent a second algebraic formulation; and
the second set of idempotent semiring operations are part of a second algebraic idempotent semiring;
obtaining a second sequence of microcode instructions, wherein the second sequence of microcode instructions are generated based on the second set of idempotent semiring operations; and
executing the second sequence of microcode instructions, wherein the set of processing units are further configured for operations based on one or more of the second algebraic formulation and the second set of idempotent semiring operations.

17. The method of claim 15, wherein:
each of the set of semiring operations comprises one or more of an associative, commutative pick operation that forms an abelian monoid and an associative tally operation that forms a monoid and distributes over the pick operation;
the associate commutative pick operation selects a value for a first plurality of values; and
the associative tally operation generates a generalized product of a second plurality of values.

18. The method of claim 15, wherein the hardware processing device comprises a systolic array and wherein the set of processing units comprises a set of data processing units.

19. The method of claim 15, wherein each processing unit of the set of processing units provides a partial result to a respective next processing unit.

20. The method of claim 19, wherein each processing unit of the set of processing units comprises a memory configured to store an operand.
